(19) United States
(12) Patent Application Publication
(10) Pub. No.: US 2014/0275293 A1
(43) Pub. Date: Sep. 18, 2014

(54) MEASUREMENT AND ANALYSIS OF MOLECULAR INTERACTIONS

(71) Applicant: The University of Vermont and State Agricultural College, Burlington, VT (US)

(72) Inventor: Bradley Palmer, Charlotte, VT (US)

(73) Assignee: The University of Vermont and State Agricultural College, Burlington, VT (US)

(21) Appl. No.: 14/207,938

(22) Filed: Mar. 13, 2014

Related U.S. Application Data
(60) Provisional application No. 61/781,877, filed on Mar. 14, 2013.

(51) Int. Cl. A61B 5/145; A61B 5/03; A61B 5/00

(52) U.S. Cl. CPC A61B 5/14546; A61B 5/14528; A61B 5/4519; A61B 5/4839; A61B 5/4848; A61B 5/72; A61B 5/036
USPC 514/789; 600/366; 600/309

(57) ABSTRACT

The present invention relates to methods for measuring molecular interactions in a viscoelastic material, as well as related products and kits. The methods involve, for instance, taking multiple pressure measurements in the viscoelastic material and calculating the complex modulus from the pressure measurements to produce the measurement of viscoelastic properties in the viscoelastic material. Also included in the invention are methods and systems for detecting molecular interactions in vivo.

### RELATED APPLICATIONS

This application claims priority under 35 U.S.C. §119 from U.S. provisional application Ser. No. 61/781,877, filed Mar. 14, 2013, the contents of which are incorporated herein in their entirety.

### FEDERALLY SPONSORED RESEARCH

This invention was made with Government support under NHLBI grant number HL59408. Accordingly, the Government has certain rights in this invention.

### BACKGROUND OF THE INVENTION

Advances in optical techniques have allowed detailed analysis of the isolated single myosin crossbridge. Upon formation of a myosin crossbridge, isomerization of the myosin molecule provides a unitary force proportional to the length of the lever arm and the extent of the lever arm swing, also called the power stroke. The time duration of crossbridge attachment, sometimes called time-on, is also measured by optical trapping. The distribution of attachment times has been found to depend upon the myosin isoform, nucleotide availability, point mutations, and several other factors. This time duration of crossbridge attachment plays a significant role in determining muscle performance observed as force and velocity of contraction at the single fiber level.

There remains, however, a considerable challenge to understand and describe how the unitary force and the temporary attachment times of myosin molecules are manifested at the level of muscle tissue, which possesses a three dimensional lattice structure and can be studied intact or submerged in physiological solutions after removal of the plasma membrane. Viscoelastic mechanics of muscle tissue represents one such macroscopic consequence of these molecular phenomena.

### SUMMARY OF THE INVENTION

Aspects of the invention relate to a quantitative justification and methodology for estimating the distribution of intermolecular reaction times. For instance the methods of the invention may be used to estimate the distribution of myosin crossbridge attachment times based on viscoelastic mechanics measured in muscle fibers.

The invention in aspects relates to a system for detecting molecular interactions in vivo. The system includes a catheter for delivering a material to a body having a distal end adapted for insertion into a body cavity in proximity of a muscle, and a proximal end, a motorized injector attached to the proximal end of the catheter such that the material can be moved from the motorized injector to the catheter, wherein the motorized injector has the capacity to inject and withdraw a material at a rapid velocity (+/−1 mL/s), a pressure detector attached near or at the distal end of the catheter, a fluid to velocity detector attached to the motorized injector, and a data collection device for collecting data from the pressure and fluid velocity detectors. In some embodiments the muscle is a ventricular chamber or vascular bed.

In some embodiments the data collection device further includes a data processing component. In some embodiments the data processing component is a Dell Computer, National Instruments PCI-6036E, IGOR 6.0. In other embodiments the data processing component includes software for calculating the myosin crossbridge lifetime value.

In some embodiments the catheter is a Solomon SIL-C70 large lumen catheter.

In other embodiments the pressure detector is pressure transducer. The pressure transducer may be a Millar SPR-1000 micro-tip.

The motorized injector includes, in some embodiments, a device to measure directly the rate of volume delivery and withdrawal, such as a TURCK In-line flow monitor. The TURCK in-line flow monitor in some embodiments is a DC Self Contained FCS-N1/2 A4P-AP8X-H1141. The motor of the motorized injector may be a modified 300 W motor.

A method for measuring the viscoelastic properties of muscle in vivo in a subject is provided according to other aspects of the invention. The method involves introducing into the subject a force that causes ventricular or vascular distention, taking multiple pressure and volume measurements, and calculating the complex modulus from the pressure and volume measurements to produce the measurement of viscoelastic properties of muscle in the subject. In some embodiments the muscle is a ventricular chamber or vascular bed. In some embodiments the force that causes ventricular or vascular distention is induced by a catheter that produces a volume change. The volume change may be a stretching of the muscle by 1%. The method may optionally be performed during cardiac catheterization.

According to other embodiments the catheter delivers 0.5-1 mL of saline with a randomized velocity to the heart.

In some embodiments the catheter is placed in the left ventricle. In other embodiments a fluid is injected into the left ventricle at the start of the isovolumic relaxation phase. The fluid is, optionally, withdrawn from the left ventricle at the start of the isovolumic relaxation phase. According to other embodiments the fluid is delivered against a pressure of 20-100 mmHg over 10-100 ms.

Multiple pressure measurements are recorded during the isovolumic relaxation phase in some embodiments. The recorded multiple pressure measurements provide a frequency-dependent viscoelasticity measurement in other embodiments.

The device is a catheter for delivering a material to a body having a distal end adapted for insertion into a body cavity, with a pressure detector positioned at or near the distal end, and a proximal end attached to a motorized injector in other embodiments.

A data collection device for collecting data from the pressure detector may be utilized according to other embodiments. The device may be any of the systems described herein.

In other aspects the invention is a method of designing a therapy for a subject by determining a value of myosin crossbridge lifetime for a subject and designing a therapy for modifying relaxation function of the subject's heart based on the value of myosin crossbridge lifetime. The method may involve treating the subject with a compound that improves relaxation function when the myosin crossbridge lifetime value is determined to be below a normal threshold. In some embodiments the step of determining a value of myosin crossbridge lifetime is achieved by the methods described herein.

A method of determining the efficacy of a therapeutic compound is provided according to other aspects of the invention. The method involves administering a therapeutic compound to a subject and determining a value of myosin crossbridge lifetime in the subject, wherein the value of myosin crossbridge lifetime following the administration of the therapeutic compound, relative to a baseline myosin crossbridge lifetime value, is determinative of the efficacy of the therapeutic compound on the relaxation potential of the muscle or cardiac smooth muscle of the subject's heart.

In other embodiments the method further involves determining the baseline myosin crossbridge lifetime value by measuring a myosin crossbridge lifetime value prior to administering the therapeutic compound to the subject. In some embodiments the step of determining a value of myosin crossbridge lifetime is achieved by any of the methods described herein.

A method for measuring molecular interactions in a viscoelastic material by taking multiple pressure measurements in the viscoelastic material and calculating the complex modulus from the pressure and volume measurements to produce the measurement of viscoelastic properties in the ventricular muscle or vascular bed is provided according to other aspects of the invention. In some embodiments the viscoelastic material is a non-living material.

The muscle may be, for instance in each of the embodiments or aspects, striated muscle.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

FIG. 1: Graph showing myosin crossbridge lifetime distribution.

FIG. 2: Graph showing left ventricular (LV) pressure and volume traces of the cardiac cycle include a relaxation interval (Diastole) that begins with isovolumic relaxation, the shaded interval of 70 ms duration at 0.4 s.

FIG. 3: Diagram of a catheter in a subject.

FIG. 4: Diagram of a system according to the invention.

FIG. 5: Set of graphs depicting the full frequency characterization of the elastic (A) and viscous (B) moduli exhibiting the C-term of Eq 1 that reflects the underlying distribution of myosin crossbridges detaching at the rate 2πc and with a mean lifetime of (2πc)⁻¹.

FIG. 6: Schematic of biological material, such as a small section of skeletal muscle, that can be placed between a length motor and force transducer. A very short segment of the muscle, dx, with cross-sectional area A contains several possibly to interacting molecules whose crosslinks allow the transfer of force across the segment. For striated muscle, one such crosslink would result from myosin of the thick filament interacting weakly (i.e., forming ionic bonds) with actin of the thin filament. Any change in length while the crosslink is attached would result in the generation of force across the segment. f(t)=force transferred across the segment by the crosslink due to strain, dL/dx, a=cross-sectional area of the crosslink, λ=elastic modulus of the crosslink, tᵢₙᵢ=time of the most recent crosslink formation, τ=duration that a crosslink has survived up to time t.

FIG. 7: Measurement of full spectrum viscoelasticity in vivo. (A). A random volume change was imposed on the left ventricular chamber on top of a sinusoidal volume change of 20 Hz. The 20 Hz signal was important to visualize whether pressure changes represented a response to the volume change or due to artifact. (B). A measured pressure tracing exhibits the elevated pressure during systole and the reduced pressure during diastole of the cardiac cycle. A 20 Hz sinusoidal waveform can be seen during systole and indicates a response to the change in volume and not an artifact. (C) and (D). The randomized signals of volume and pressure were taken over 0.5 s intervals (with a Hanning window focused on the central 0.25 s) and used to calculate the elastic (C) and viscous (D) moduli during systole and diastole. As can be seen in these figures, an elevated viscoelastic response is indicated during systole, as would be expected. The full spectrum viscoelasticity is detected over the frequency range of ˜2-250 Hz.

FIG. 8: Photograph of a device of the invention.

### DETAILED DESCRIPTION

A few groups have attempted to measure viscoelastic properties of the left ventricle by detecting change in LV pressure or circumference (Honda et al., 1994; Encke et al., 1996; Starc et al., 1996; Kim et al., 2000; Campbell et al., 2005; Campbell et al., 2008). However, those methods and measures provided only a generalized measure of stiffness or viscosity (force/length and force/velocity), rather than a full spectrum measurement. The methods of the invention provide a full spectrum characterization of the viscoelastic properties of a variety of materials. Based on this measurement, analysis of micro-interactions such as myosin crossbridge formation can to be achieved.

The invention in aspects relates to the measurement of viscoelastic properties of materials involving interactions between 2 or more molecular components. For example, the viscoelastic properties of the left ventricle during isovolumic filling period of diastole may be measured. Briefly, the device or system (10, FIG. 4) consists of a motorized injector (40) on the end of a catheter (20) and a pressure sensor (30) fitted onto the same catheter. The injector moves rapidly to effectively release liquid and increase the volume of the tissue at the other end of the catheter, such as the ventricle. The pressure sensor records the change in tissue i.e. ventricle pressure arising from the change in volume. A frequency domain analysis of the volume and pressure transients provides a measure of the viscoelastic properties of the tissue i.e., ventricular wall. These viscoelastic properties are then used to calculate the number of reactions, i.e., myosin crossbridges×crossbridge stiffness attached during diastole and the rate at which these crossbridges would be detaching. Such a measure could provide important information regarding the source (prolonged activation of thin filament vs. prolonged lifetime of a crossbridge vs. passive stiffness) of diastolic dysfunction.

The methodology of the invention has demonstrated that viscoelastic mechanics of striated muscle, measured as elastic and viscous moduli, emerge directly from the myosin crossbridge attachment time, tₐₜₜ, also called time-on. The distribution of tₐₜₜ was modeled using a single exponential distribution (Palmer et al., 2007) and also a gamma distribution with shape parameter, p, and scale parameter, β (Palmer et al., 2011). At 5 mM MgATP, β was similar between mouse α-MyHC (16.0±3.7 ms) and β-MyHC (17.9±2.0 ms), and p was higher (P<0.05) for β-MyHC (5.6±0.4 no units) compared to α-MyHC (3.2±0.9). At 1 mM MgATP, p approached a value of 10 in both isoforms, but β rose only in the β-MyHC (34.8±5.8 ms). The estimated mean tₐₜₜ (i.e., pβ product) was longer in the β-MyHC compared to α-MyHC, and became prolonged in both isoforms as MgATP was reduced as expected. The application of the viscoelastic model to these isoforms and varying MgATP conditions shows that tₐₜₜ is better modeled as a gamma distribution due to its representing multiple temporal events occurring within tₐₜₜ compared to a single exponential distribution which assumes only one temporal event within tₐₜₜ (Palmer et al., 2011).

Measuring viscoelastic properties of tissue such as the left ventricle has been to attempted before (Honda et al., 1994; Encke et al., 1996; Starc et al., 1996; Kim et al., 2000; Campbell et al., 2005; Campbell et al., 2008). However, these measures have previously been made only in isolated animal hearts using balloons or saline that fill the ventricle. The system of the invention is able for the first time to provide a full spectrum of viscoelastic characteristics, which is required to calculate myosin crossbridge lifetimes in a living animal. In some instances it is desirable to perform the measurement during isovolumic relaxation when both valves are closed.

The system and methods of the invention are described herein with the specific focus of a medical diagnostic tool for detecting the contribution of specific molecules (i.e., myosin) to poor relaxation of a beating heart. However, this description is exemplary to provide detailed analysis of the system. The system and methodology however is not limited to the heart. It may be used to measure viscoelastic properties of any material and optionally any accessible material in vivo. Relaxation of the heart muscle is critical for insuring that sufficient blood volume fills the chambers between each heartbeat. The number of patients in the US that suffer from poor or insufficient relaxation function is in the 10's of millions. The methods and systems of the invention provide a new diagnostic tool for evaluating the molecular basis of poor relaxation function in millions of patients. The information provided by the methodology is then used to better tailor treatments for these patients.

The methods broadly involve (i) measuring viscoelasticity of heart muscle in a living subject and (ii) applying a mathematical algorithm to reveal the temporal properties of underlying molecular events, which are known to be due to the force-producing molecule myosin, resulting in the detection of the specific molecular level events that contribute to inhibition of heart muscle relaxation.

Heart muscle may lend itself to this sort of analysis because the most common molecular events are overwhelmingly due to a single molecular species, the myosin enzyme. The myosin enzyme forms a molecular bond, called a crossbridge, with a neighboring filamentous molecule, actin, and then generates force Millions of these force-producing myosin crossbridges are formed during muscle contraction. The myosin crossbridge is temporary and, because it is an enzyme, its lifetime is dictated by its biochemical properties and surrounding environment. The lifetime of a myosin to crossbridge, however, is not always the same and can be considered a stochastic (or random) process such that a distribution (or probability) of lifetimes better describes the length of time any one myosin crossbridge remains formed. FIG. 1 shows an example distribution of lifetimes. The mean crossbridge lifetime is ˜10 ms for humans. A shorter mean lifetime means a faster rate of crossbridge detachment from actin and a faster rate of pressure drop during the relaxation phase of the cardiac cycle.

Using the molecular level information obtained from the macroscopic measures of viscoelasticity, the medical community will have a tool for detecting the molecular-level consequences of disease states and for evaluating the efficacy of therapies. The use of viscosity to detect molecular-level events is also useful in other fields, such as metallurgy, cement and adhesives, variable viscous fluids, plastics production and other materials-based markets.

Until now, measures of molecular events (for instance using optical trapping methods) would have to detect single molecular events one at a time. It takes weeks to collect enough data to estimate the result of many events and produce the distribution of lifetimes like that in FIG. 1. And the heart of origin is destroyed in the process. The invention provides essentially the same information within a matter of minutes and within a living subject.

The examples described herein are designed to measure a frequency-dependent viscoelasticity of the left ventricle during the isovolumic relaxation phase of the cardiac cycle. The isovolumic relaxation phase is a roughly 70 ms time period when the heart valves are closed, blood does not enter or leave the left ventricle and blood volume is constant (see FIG. 2). During this time period, the heart muscle is relaxing and the pressure in the left ventricle is dropping just prior to filling with blood. At the molecular level, relaxation relies upon the inhibition of new myosin crossbridges being formed and the rate at which any pre-existing myosin crossbridges will now detach from filamentous actin. The contraction force is diminishing at a rate directly related to rate at which myosin crossbridges detach, i.e., at a rate of (10 ms)⁻¹ or 100 s⁻¹. This rate of myosin crossbridge detachment must be fast enough to insure a relaxed and flaccid state of the muscle prior to the left atrium filling the left ventricle with blood. Any crossbridges that do not detach and remain formed during this time period will slow the relaxation process and prevent adequate filling once the mitral valve opens.

To measure the viscoelasticity of the left ventricle during the isovolumic relaxation phase, a specialized system has been developed including a catheter placed into the left ventricular chamber (see FIG. 3). Catherization is relatively common for cardiac patients undergoing evaluation of coronary artery disease (˜2.7 million per year in US). Evaluation of left ventricular pressure as shown in FIG. 3 can accompany this procedure.

A schematic diagram of the system of the invention is shown in FIG. 4. The system (10) is designed for detecting molecular interactions in vivo. The system includes a catheter (20) for delivering a material to a body having a distal end (22) adapted for insertion into a body cavity in proximity of a striated muscle, and a proximal end (24). It also includes a motorized injector (40) attached to the proximal end (24) of the catheter (20) such that the material can be moved from the motorized injector (40) to the catheter (20), wherein the motorized injector (40) has the capacity to inject and withdraw a material at a rapid velocity. A pressure detector (30) is attached near or at the distal end of the catheter (22). The system also includes a data collection device (50) for collecting data from the pressure detector. A computer, monitor or other type of work station (60) may also be included as part of the system.

The catheter may be any catheter used in a catheter based medical procedure (e.g., percutaneous intervention procedures and therapeutic delivery procedures). In fact the catheter may serve a dual purpose during a patient procedure, where it is used for both the method of the invention and another medical procedure during the same placement in a patient. For example, the catheter may be simultaneously used in a diagnostic procedure, such as where a contrast media is injected into one or more coronary arteries through a catheter and an image of the patient's heart is taken. Other catheter based medical procedures may also include balloon angioplasty, stent placement, treatment of peripheral vascular disease. The specific type of catheter thus may be selected based on the type of procedure that is to be co-performed with the method of the invention.

The catheter typically has an elongated hollow tube having a proximal end, a distal end, and a lumen along the length of the catheter. The proximal end of the catheter is connected to either tubing or directly to the motorized injector. The tubing if present is in fluid communication with a vessel within the injector.

The injector is preferably a motorized injector which is capable of carefully controlling the speed, volume and pressure of the injected fluid. The system of the invention provides a small but rapid volume change to the chamber so that the muscle is stretched on the order of 1% in circumference very quickly. Typically, 0.5-1 mL of saline is injected with a randomized velocity and then the pressure during the isovolumic relaxation phase is detected in the beating heart of an animal.

Typically the method involves delivery of 0.5-1 mL liquid with a randomized velocity against a pressure of 20-100 mmHg over 10-100 ms. This is also accomplished with a motorized injector such as a plunger driven by a high speed motor. The motorized injector may include a plunger detector, which permits detection of volume delivery with high precision.

The continuous and randomized velocity is used to assure that a specialized feedback control is not needed to time an injection to hit the 70 ms isovolumic relaxation phase. The randomized velocity will also assure a volume signal consisting of a broad range of frequencies. The pressure signal in response will likewise have a broad range of frequencies.

The system of the invention also includes a pressure sensor (30). A Millar pressure transducer is one example of a pressure sensor that is useful according to the invention. These types of pressure transducers have been used previously for recording left ventricular pressures and have demonstrated sufficient fidelity and frequency response for the methods described herein. Typically the pressure sensor operably coupled to the catheter, wherein the at least one pressure sensor operates to provide the user with pressure and/or volume data.

During the isovolumic relaxation phase, the measures of pressure and volume will provide a frequency-dependent viscoelasticity for the entire left ventricular chamber, which will include the viscoelastic contributions of the left ventricular muscle, valves and blood. This frequency-dependent viscoelasticity is also called the Complex Modulus, Y(ω), which is calculated in frequency space as the ratio of the Fourier Transform of the pressure change divided by the Fourier Transform of the volume change. Randomized velocities were used to calculate the Complex Modulus.

The Complex Modulus for human left ventricular muscle, in terms of its real (elastic) and imaginary (viscous) parts, is illustrated in FIG. 5. The rate at which myosin crossbridges detach is related to the Complex Modulus in the C-term of Eq 1:

$$Y ( ω ) = {A ( i ω )}^{k} - B ( \frac{i ω}{2 π b + i ω} ) + C ( \frac{i ω}{2 π c + i ω} ) .$$

While the net viscoelasticity includes the contributions of muscle, valves and blood, only the muscle contains myosin, and only myosin produces a signature frequency dependent viscoelasticity like that shown in FIG. 5 and described by the C-term of Eq. 1. The mean myosin crossbridge lifetime is calculated as (2πc)⁻¹ after fitting Eq. 1 to the recorded Complex Modulus (Palmer et al., Biophys J, 2007; Palmer et al., J Biomed Biotechnol, 2011). This C-term signature can only occur with the myosin enzyme and cannot be due to other molecular events of the blood or valves. The so-called sinusoidal analysis method for producing the complex modulus with high fidelity is shown in FIG. 5.

The methods shown in the examples provide an analysis of the viscoelastic signature of myosin crossbridges i.e. such as that shown in FIG. 5. However, the methods of the invention form the basis of a new diagnostic tool for evaluating the underlying cause of poor relaxation function, as well as for analyzing the viscoelastic properties of other materials. Currently, physicians often apply the term ‘left ventricular stiffness’ to refer to the left ventricular resistance to filling. However, with the methods of the invention, the practice of cardiology has the opportunity to consider the specific molecular-level mechanical events that dictate left ventricular stiffness. This methodology serves as a tool to diagnose the contribution of myosin crossbridge lifetime to poor relaxation and as a tool to evaluate the effectiveness of treatments designed to shorten crossbridge lifetime and thereby improve relaxation function.

The system of the invention may be manually controlled or may have robotic components. A robotic system may be any system configured to allow a user to perform the catheter-based procedures of the invention via a robotic system by operating various controls from a workstation (60). The workstation (60) may include a user interface and controls. Controls allow the user to control the system (10) to perform the methods of the invention. For example, controls may be configured to cause the system to perform various tasks, such as controlled injection of a liquid, advancing, retracting, or rotating a catheter, or to perform any other function that may be performed as part of a catheter based medical procedure. The catheter may be steerable, and controls may be configured to allow a user to steer the catheter from a remote workstation. For instance, the user may control the bending of the distal tip of a steerable catheter.

The system may further include one or more monitors. Monitors may be configured to display information or patient specific data to the user. For example, a monitor may be configured to display pressure data, image data (e.g., x-ray images, MRI images, CT images, ultrasound images, etc.), hemodynamic data (e.g., blood pressure, heart rate, etc.), patient record information (e.g., medical history, age, weight, etc.). In addition, a monitor may be configured to display procedure specific information (e.g., duration of procedure, catheter or guide wire position, volume of liquid delivered, etc.). The monitor may be configured to display information regarding the position and/or bend of the distal tip of a steerable catheter. The monitor in some embodiments may be connected to or associated with the data collection device (50).

Communication between the various components of the system (10) may be accomplished via communication links. Communication links may be dedicated wires or wireless connections. Communication links may also represent communication over a network. The system may be connected or configured to include any other systems and/or devices not explicitly shown. For example, the system may include IVUS systems, image processing engines, data storage and archive systems, automatic balloon and/or stent inflation systems, contrast media and/or medicine injection systems, medicine tracking and/or logging systems, user logs, encryption systems, systems to restrict access or use of the system.

The positioning of the pressure transducer and the catheter into a patient's ventricle includes the steps of advancing the distal end of the catheter into the patient's ventricle, steering the distal end of the catheter within the ventricle optionally by expanding an expandable member at the distal end of the catheter to move the tip of the catheter within the ventricle. The method may be alternatively used to deliver the pressure sensor to any other suitable chamber or organ of a patient or in any other suitable environment, and for any suitable purpose.

The step of advancing the distal end of a catheter into the patient's ventricle functions to position the distal end of the catheter into the ventricle such that the catheter and pressure sensor are positioned to take the measurements necessary for the methodology. In some embodiments, the distal portion of the catheter may be advanced through the aorta and aortic valve, and into the left ventricle. Additionally, the distal portion of the catheter may first be advanced percutaneously into the patient's vasculature, and then advanced through the vasculature to the aorta, or any other suitable vessel. In some embodiments, the distal portion of the catheter may be advanced into the ventricle through the distal wall of the ventricle.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Introduction: The Calculations Presented Below in Example 1 are Included in Palmer et al J. Biomedicine and Biotech, v. 2011, p.1, 2011

### Optical Techniques

Optical techniques have allowed detailed analysis of the isolated single myosin crossbridge. Upon formation of a myosin crossbridge, isomerization of the myosin molecule provides a unitary force proportional to the length of the lever arm and the extent of the lever arm swing, also called the power stroke. The time duration of crossbridge attachment, sometimes called time-on, is also measured by optical trapping. The distribution of attachment times has been found to depend upon the myosin isoform, nucleotide availability, point mutations, and several other factors. This time duration of crossbridge attachment plays a significant role in determining muscle performance observed as force and velocity of contraction at the single fiber level.

### Length Perturbation Analysis of Muscles

Length perturbation analysis of muscles provides a means to quantify viscoelastic mechanics and entails applying a small length change at one end of a muscle and recording the force response on the other end as illustrated in FIG. 6. The length perturbation and force response are then subjected to Fourier transformation, and the dynamic mechanical properties of the muscle are characterized by the complex ratio of the transformed force response normalized to cross-sectional area (or σ(ω)=Fourier transformed tensile stress) divided by the transformed length perturbation normalized to initial length (or ε(ω)=Fourier transformed strain). This complex ratio is also called the mechanical transfer function or complex modulus of the muscle, Y(ω)=σ(ω)/ε(ω). The real and imaginary parts are, respectively, termed the elastic modulus and viscous modulus of the muscle.

The frequency characteristic dips (low values) and shoulders (high values) observed in the elastic and viscous moduli of activated muscle are sensitive to species of origin, myosin isoform, and varying concentrations of MgATP and therefore reflect the macroscopic consequence of myosin crossbridge kinetics. The dips in the moduli, most notably the negatively valued viscous modulus, occur near the frequencies at which the muscle operates in vivo. The shoulders of the moduli, however, are more prominent in magnitude. According to the previous modeling endeavor, the shoulders appearing at these higher frequencies of the moduli reflect the mechanical consequences of intermittent myosin crossbridge formation. In that work, it was proposed that a two-state model of the acto-myosin crossbridge governed by first-order kinetics gives rise to a viscoelastic work-absorbing property, termed the C-process by Kawai and colleagues, which is characterized by an exponential rate constant equivalent to the myosin crossbridge off rate termed g. The mean myosin attachment time, based on a single exponential distribution of attachment times, could be estimated as the reciprocal of this exponential rate constant, 2πc, after fitting (1) to a measured complex modulus:

$$Y ( ω ) = {A ( i ω )}^{k} - B ( \frac{i ω}{2 π b + i ω} ) + C ( \frac{i ω}{2 π c + i ω} ) . \qquad ( 1 )$$

While the single exponential representation of C-process and its interpretation have been valuable for examining a variety of muscle types under a number of conditions, the assumptions of first-order kinetics and a single exponential distribution of myosin attachment times are limiting. Multiple time periods associated with multiple biochemical states make up the myosin crossbridge cycle. These multiple states constitute the entire myosin crossbridge attachment time. Such an addition of multiple smaller time periods would result in a multiple exponential distribution or a gamma distribution of the crossbridge attachment times rather than a single exponential distribution assumed by first-order kinetics.

An examination showed that the mechanical consequences of the distribution of myosin attachment times are represented by a gamma distribution, which allows the description of more discrete and longer lived time-on and yet also allows for the possibility of a single exponential function. The consideration of the gamma distribution effectively posed the hypothesis that multiple temporal events which occur within the time of attachment can be discerned in the viscoelastic response of striated muscle at the macroscopic level. An analytical solution was found to the mechanical consequences that emerge as the shoulders of the elastic and viscous moduli. The validity of the analytical solution was also demonstrated with computer simulations. Finally, it was demonstrated that the application of the gamma distribution representation of the C-process in a comparison of mouse cardiac myosin heavy chain isoforms, α-MyHC and β-MyHC, subjected to varying concentrations of MgATP.

### Material and Methods

Viscoelastic Mechanics

All procedures were reviewed and approved by the Institutional Animal Care and Use Committees of The University of Vermont. Male wild-type mice were fed either a normal mouse diet (WT) or an iodine deficient, 0.15% 6-N-propyl-2-thiouracil (PTU) diet for at least nine weeks prior to their being killed by rapid cervical dislocation. The PTU mice therefore became hypothyroid resulting in the expression β-MyHC in the myocardium in contrast to the α-MyHC expressed in the WT mouse heart.

Mouse left ventricular skinned myocardial strips were prepared using methods similar to those described previously to yield thin strips (˜140 μm diameter, ˜800 μm length) with longitudinally oriented parallel fibers. These strips were chemically skinned for 2 hr at 22° C. and stored at −20° C. for no more than 5 days. At the time of study, aluminum T-clips were attached to the ends of a strip ˜150 μm apart. The strip was mounted between a piezoelectric motor (Physik Instrumente, Auburn, Mass.) and a strain gauge (SensoNor, Horten, Norway), lowered into a 30 μL droplet of relaxing solution maintained at 37° C. and incrementally stretched to and maintained at 2.2 μm sarcomere length detected by videography and digital Fourier transform techniques (IonOptix, Milton, Mass.).

Strips were calcium activated at pCa 4.5 and subjected to decreasing concentrations of 5, 2, and 1 mM MgATP by exchanging equal volumes of rigor to solution. Sinusoidal perturbations of amplitude 0.125% strip length were applied over the frequency range 0.125-250 Hz. The elastic and viscous moduli were calculated from the recorded tension transient as the relative magnitudes of the in-phase and out-of-phase components with respect to the imposed sinusoidal length perturbations. The measured complex modulus was fit to (1), representing the single exponential distribution model of tₐₜₜ, and to (2) below, representing the gamma distribution model of tₐₜₜ, using a non weighted Levenburg-Marquardt non linear least-squares routine running within IDL Version 7.0 (ITT, Boulder, Colo.)

$$Y ( ω ) = {A ( i ω )}^{k} - B ( \frac{i ω}{2 π b + i ω} ) + ξ \{ 1 - \frac{1}{p i ω β} ( \frac{{( i ω β + 1 )}^{p} - 1}{{( i ω β + 1 )}^{p}} ) \} . \qquad ( 2 )$$

Mean myosin time attached was calculated as (2πc)⁻¹ when the fit was performed with (1) and as pβ (or fits using (2). The correlation coefficients between the recorded data and the fitted models were calculated as a Pearson correlation coefficient. The two models used to calculate the mean myosin time attached were compared using linear correlation. Data points are presented as mean±sem.

All human subjects signed informed consent forms approved by the Institutional Review Board of the University of Vermont prior to biopsy. Human skeletal muscle and cardiac muscle were biopsied as described previously (Donaldson et al. 2012 or Selby et al. 2011). Skeletal muscle was bathed in low calcium concentration (pCa 8) and 30 mM BDM, which inhibited the formation of strongly bound myosin crossbridges [30]. Other constituents in the bath were (in mM): MARK . . . Viscoelastic properties of relaxed skeletal muscle were detected using sinusoidal length perturbations with amplitudes of 0.25%, which were small enough to assure a linear viscoelastic response. Hydration was reduced by addition of 0.34% wt/vol Dextran-T10 (molecular weight 10 kDa). Osmotic compression of the myofilament lattice was achieved by addition of 4% wt/vol Dextran-T500 (molecular weight 500 kDa), which matched the hydration conditions of the 0.34% wt/vol Dextran-T10. Temperature was varied from 15° C. to 25° C. All skeletal muscle fibers were subsequently identified for fiber-type and myosin isoform, and only results from slow skeletal muscle are presented here. Cardiac muscle was not typed; approximately 95% of myosin in the human myocardium is the same isoform as human to slow skeletal muscle. Rigor conditions were achieved by removing MgATP from the bathing solution. Ionic strength was altered by reducing NaMS concentration to 40 mM.

Results

It was demonstrated that viscoelastic mechanics of striated muscle, measured as elastic and viscous moduli, emerge directly from the myosin crossbridge attachment time, tₐₜₜ, also called time-on. The distribution of tₐₜₜ was modeled using a gamma distribution with shape parameter, p, and scale parameter, β. At 5 mM MgATP, β was similar between mouse α-MyHC (16.0±3.7 ms) and β-MyHC (17.9±2.0 ms), and p was higher (P<0.05) for β-MyHC (5.6±0.4 no units) compared to α-MyHC (3.2±0.9). At 1 mM MgATP, p approached a value of 10 in both isoforms, but β rose only in the β-MyHC (34.8±5.8 ms). The estimated mean tₐₜₜ (i.e., pβ product) was longer in the β-MyHC compared to α-MyHC, and became prolonged in both isoforms as MgATP was reduced as expected. The application of the developed viscoelastic model to these isoforms and varying MgATP conditions shows that tₐₜₜ is better modeled as a gamma distribution due to its representing multiple temporal events occurring within tₐₜₜ compared to a single exponential distribution which assumes only one temporal event within tₐₜₜ.

FIG. 6 is a schematic of biological material, such as a small section of skeletal muscle, that can be placed between a length motor and force transducer. A very short segment of the muscle, dx, with cross-sectional area A contains several possibly interacting molecules whose crosslinks allow the transfer of force across the segment. For striated muscle, one such crosslink would result from myosin of the thick filament interacting weakly (i.e., forming ionic bonds) with actin of the thin filament. Any change in length while the crosslink is attached would result in the generation of force across the segment. f(t)=force transferred across the segment by the crosslink due to strain, dL/dx, a=cross-sectional area of the crosslink, λ=elastic modulus of the crosslink, tᵢₙᵢ=time of the most recent crosslink formation, τ=duration that a crosslink has survived up to time t.

### Example 2

### In Vivo Measurement of Full Spectrum Viscoelasticity

The methods of the invention were tested in vivo. The results of the analysis are shown in FIG. 7. A random volume change was imposed on the left ventricular chamber on top of a sinusoidal volume change of 20 Hz. The 20 Hz signal was important to visualize whether pressure changes represented a response to the volume change or due to artifact. This is depicted in FIG. 7A. A measured pressure tracing exhibits the elevated pressure during systole and the reduced pressure during diastole of the cardiac cycle, as shown in FIG. 7B. A 20 Hz sinusoidal waveform can be seen during systole and indicates a response to the change in volume and not an artifact. The randomized signals of volume and pressure were taken over 0.5 s intervals (with a Hanning window focused on the central 0.25 s) and used to calculate the elastic (FIG. 7C) and viscous (FIG. 7D) moduli during systole and diastole. The units of the moduli are arbitrary as the true dimensions of displacement were not calculated. The data in FIG. 7 demonstrates that an elevated viscoelastic response is indicated during systole, as would be expected. The full spectrum viscoelasticity is detected over the frequency range of ˜2-250 Hz.

FIG. 8 is a photograph of a device of the invention used to conduct in vivo studies such as the one described herein with the data shown in FIG. 7.

### REFERENCES

Honda, Hideyuki, Yoshiro Koiwa, Kazuhiko Takagi, Jun-Ichi Kikuchi, Nobuo Hoshi, Tamotsu Takishima, and James P. Butler. Noninvasive measurement of left ventricular myocardial elasticity. Am. J. Physiol. 266 (Heart Circ. PhysioE. 35): H881-H890, 1994.
Torsten Encke, Bert Flemming, Dieter Roloff, Thomas Wronski. Ventricular volume elasticity: a component of cardiovascular control? Journal of the Autonomic Nervous System 57:173-176, 1996.
Start, Vito, Edward L. Yellin, and Srdjan D. Nikolic. Viscoelastic behavior of the isolated guinea pig left ventricle in diastole. Am. J. Physiol Heart Circ Physiol 271: H1314-H1324, 1996.
Borut Kim Nejka Pototnik Vito Starc. The reversible displacement of the passive diastolic P-V curve in the isolated guinea pig left ventricle. Pfluegers Arch—Eur J Physiol 439 [Suppl]: R206-R207, 2000.
Campbell, K. B., Y. Wu, A. M. Simpson, R. D. Kirkpatrick, S. G. Shroff, H. L. Granzier, and B. K. Slinker. Dynamic myocardial contractile parameters from left ventricular pressure volume measurements. Am J Physiol Heart Circ Physiol 289: H114-H130, 2005.
Campbell K B, Simpson A M, Campbell S G, Granzier H L, Slinker B K. Dynamic left ventricular elastance: a model for integrating cardiac muscle contraction into ventricular pressure-volume relationships. J Appl Physiol 104: 958-975, 2008.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A system for detecting molecular interactions in vivo, comprising
a catheter for delivering a material to a body having a distal end adapted for insertion into a body cavity in proximity of a muscle, and a proximal end,
a motorized injector attached to the proximal end of the catheter such that the material can be moved from the motorized injector to the catheter, wherein the motorized injector has the capacity to inject and withdraw a material at a rapid velocity (+/−1 mL/s),
a pressure detector attached near or at the distal end of the catheter,
a fluid velocity detector attached to the motorized injector, and
a data collection device for collecting data from the pressure and fluid velocity detectors.

2. The system of claim 1 wherein the muscle is a ventricular chamber or vascular bed.

3. The system of claim 1, wherein the data collection device further includes a data processing component.

4. The system of claim 1, wherein the catheter is a SolomonSIL-C70 large lumen catheter.

5. The system of claim 1, wherein the pressure detector is pressure transducer.

6. The system of claim 4, wherein the pressure transducer is a MillarSPR-1000 micro-tip.

7. The system of claim 1, wherein the motorized injector includes a device to measure directly the rate of volume delivery and withdrawal.

8. The system of claim 8, wherein the device to measure directly the rate of volume delivery and withdrawal is a TURCK In-line flow monitor.

9. The system of claim 8, wherein the TURCK in-line flow monitor is a DC Self Contained FCS-N1/2 A4P-AP8X-H1141.

10. The system of claim 1, wherein the motor of the motorized injector is a modified 300 W motor.

11. The system of claim 1, wherein the data processing component is a Dell Computer, National Instruments PCI-6036E, IGOR 6.0.

12. The system of claim 1, wherein the data processing component includes software for calculating the myosin crossbridge lifetime value.

13. A method for measuring the viscoelastic properties of muscle in vivo in a subject, comprising:
introducing into the subject a force that causes ventricular or vascular distention, taking multiple pressure and volume measurements, and calculating the complex modulus from the pressure and volume measurements to produce the measurement of viscoelastic properties of muscle in the subject.

14. The system of claim 1 wherein the muscle is a ventricular chamber or vascular bed.

15. The method of claim 13, wherein the force that causes ventricular or vascular distention is induced by a catheter that produces a volume change.

16. The method of claim 15, wherein the volume change is a stretching of the muscle by 1%.

17-27. (canceled)

28. A method of designing a therapy for a subject comprising,
determining a value of myosin crossbridge lifetime for a subject and designing a therapy for modifying relaxation function of the subject's heart based on the value of myosin crossbridge lifetime.

29. The method of claim 28, further comprising treating the subject with a compound that improves relaxation function when the myosin crossbridge lifetime value is determined to be below a normal threshold.

30. (canceled)

31. A method of determining the efficacy of a therapeutic compound comprising,
administering a therapeutic compound to a subject and determining a value of myosin crossbridge lifetime in the subject, wherein the value of myosin crossbridge lifetime following the administration of the therapeutic compound, relative to a baseline myosin crossbridge lifetime value, is determinative of the efficacy of the therapeutic compound on the relaxation potential of the muscle of the subject's heart.

32. The method of claim 31, further comprising determining the baseline myosin crossbridge lifetime value by measuring a myosin crossbridge lifetime value prior to administering the therapeutic compound to the subject.

33-35. (canceled)
